(19) United States
(12) Patent Application Publication
(10) Pub. No.: US 2026/0216289 A1
(43) Pub. Date: Jul. 30, 2026

(54) USE OF RECOMBINANT FIBRINOGEN-LIKE DOMAIN OF ANGIOPOIETIN-LIKE 4 FOR TREATING ADVERSE POST-ISCHEMIC CARDIAC REMODELING IN A PATIENT WHO EXPERIENCED A MYOCARDIAL INFARCTION

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS, Paris (FR); CENTRE NATIONAL DE LA RECHERCHE SCIENTIFIQUE (CNRS), Paris (FR); COLLÈGE DE FRANCE, Paris (FR); ECOLE NATIONALE VÉTÉRINAIRE D'ALFORT, Maisons-Alfort (FR); UNIVERSITÉ PARIS-EST CRÉTEIL VAL DE MARNE, Creteil (FR)

(72) Inventors: Stéphane GERMAIN, Paris (FR); Bijan GHALEH-MARZBAN, Creteil (FR)

(21) Appl. No.: 19/145,073

(22) PCT Filed: Jan. 8, 2024
(86) PCT No.: PCT/EP2024/050259

§ 371 Date: Jul. 1, 2025

(30) Foreign Application Priority Data
Jan. 9, 2023 (EP) 23305023.6

(51) Int. Cl. A61K 38/18; A61K 31/7105; A61P 9/10

(52) U.S. Cl. CPC A61K 38/1891; A61K 31/7105; A61P 9/10

(57) ABSTRACT

Ischemic heart diseases are leading causes of death and reduced life quality worldwide. Although revascularization strategies significantly reduce mortality after acute myocardial infarction (MI), a large number of patients with MI develop chronic heart failure over time. The inventors previously reported that human recombinant ANGPTL4 counteracts ischemia-induced vascular endothelial growth factor signaling and disruption of endothelial cell junctions thus inhibiting vascular permeability. When administered before MI, the inventors could show that ANGPTL4 leads to protection of the coronary capillary network, reduction of no-reflow and infarct size in mice. The inventors also showed that the therapeutic effects observed with ANGPTL4 in ischemic conditions are not due to the CCD fragments but are brought by the FLD fragment (WO2016/110498). To further test the therapeutic potential of the FLD of ANGPTL4 at the onset of reperfusion, the inventors here used a pig model in a clinically relevant model of acute myocardial infarction which can be easily and safely translated to patient treatment. The inventors demonstrated that local (antegrade) delivery of FLD ANGPTL4 to the infarcted pig heart can target the affected regions in an efficient and clinically relevant manner. A single dose of FLD of ANGPTL4 improved heart function, infarct size, fibrosis, and adverse remodeling parameters 28 days after MI. Short term MI experiments along with complementary murine studies revealed myocardial protection. Thus, a single dose of FLD of ANGPTL4 is capable of reducing ischemia-reperfusion injury and protects against deleterious post-ischemic cardiac remodeling and consecutive ischemic heart failure.

### FIELD OF THE INVENTION

The present invention is in the field of medicine, in particular cardiology.

### BACKGROUND OF THE INVENTION

Myocardial infarction (MI), the most prevalent manifestation of cardiovascular diseases, is associated with high mortality and morbidity (Mortality, G. B. D. and C. Causes of Death, Global, regional, and national age-sex specific all-cause and cause-specific mortality for 240 causes of death, 1990-2013: a systematic analysis for the Global Burden of Disease Study 2013. Lancet, 2015. 385(9963): p. 117-71). Nevertheless, considerable advances have been achieved in the early management of acute coronary thrombotic occlusion, including rapid mechanical restauration of coronary artery blood flow and anti-platelet therapies (Frangogiannis, N. G., The inflammatory response in myocardial injury, repair, and remodeling. Nat Rev Cardiol, 2014. 11(5): p. 255-65). A marked decline in early mortality of patients with MI has been observed over the last decades (Puymirat, E., et al., Association of changes in clinical characteristics and management with improvement in survival among patients with ST-elevation myocardial infarction. JAMA, 2012. 308(10): p. 998-1006). However, long term effects of ischemia-related cardiac damage continue to be a clinical and social burden, due to increased risk of arrhythmias, heart failure and repetitive hospitalizations (Lavoie, L., et al., Burden and Prevention of Adverse Cardiac Events in Patients with Concomitant Chronic Heart Failure and Coronary Artery Disease: A Literature Review. Cardiovasc Ther, 2016. 34(3): p. 152-60). Moreover, more efforts have to be deployed towards the development of therapeutic approaches targeting pathophysiological pathways involved in post-ischemic cardiac remodeling. Human recombinant ANGPTL4 counteracts ischemia-induced vascular endothelial growth factor signaling and disruption of endothelial cell junctions thus inhibiting vascular permeability (Galaup, Ariane, et al. “Protection against myocardial infarction and no-reflow through preservation of vascular integrity by angiopoietin-like 4.” Circulation 125.1 (2012): 140-149). When administered before MI, ANGPTL4 leads to protection of the coronary capillary network, reduction of no-reflow and infarct size (WO2011/089152 and WO2016/110498). Then, studies showed that a decrease of infarct size does not correlate with an improvement in ventricular remodeling (Piot C et al., 2008, N Engl J Med & Cung T-T et al., 2015, N Engl J Med). Therefore, the effects of ANGPTL4 on post-ischemic cardiac remodeling have never been investigated.

### SUMMARY OF THE INVENTION

The present invention is defined by the claims. In particular, the present invention relates to the use of the recombinant fibrinogen-like domain of angiopoietin-like 4 for treating adverse post-ischemic cardiac remodeling in a patient who experienced a myocardial infarction.

### DETAILED DESCRIPTION OF THE INVENTION

Ischemic heart diseases are leading causes of death and reduced life quality worldwide. Although revascularization strategies significantly reduce mortality after acute myocardial infarction (MI), a large number of patients with MI develop chronic heart failure over time. The inventors previously reported that human recombinant ANGPTL4 counteracts ischemia-induced vascular endothelial growth factor signaling and disruption of endothelial cell junctions thus inhibiting vascular permeability. When administered before MI, the inventors could show that ANGPTL4 leads to protection of the coronary capillary network, reduction of no-reflow and infarct size in mice. The inventors also showed that the therapeutic effects observed with ANGPTL4 in ischemic conditions are not due to the CCD fragments but are brought by the FLD fragment (WO2016/110498). To further test the therapeutic potential of the FLD of ANGPTL4 at the onset of reperfusion, the inventors here used a pig model in a clinically relevant model of acute myocardial infarction which can be easily and safely translated to patient treatment. The inventors demonstrated that local (antegrade) delivery of FLD ANGPTL4 to the infarcted pig heart can target the affected regions in an efficient and clinically relevant manner. A single dose of FLD of ANGPTL4 improved heart function, infarct size, fibrosis, and adverse remodeling parameters 28 days after MI. Short term MI experiments along with complementary murine studies revealed myocardial protection. Thus, a single dose of FLD of ANGPTL4 is capable of reducing ischemia-reperfusion injury and protects against deleterious post-ischemic cardiac remodeling and consecutive ischemic heart failure. Moreover, the inventors using intracoronary injection showed a decrease of the injected product diffusion to other organs.

### Main Definitions

As used herein, the term “polypeptide” has its general meaning in the art and refers to a polymer of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

As used herein, the term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In some embodiments, the polynucleotide comprises an mRNA. In other aspect, the mRNA is a synthetic mRNA. In some embodiments, the synthetic mRNA comprises at least one unnatural nucleobase. In some embodiments, all nucleobases of a certain class have been replaced with unnatural nucleobases (e.g., all uridines in a polynucleotide disclosed herein can be replaced with an unnatural nucleobase, e.g., 5-methoxyuridine). In some embodiments, the polynucleotide (e.g., a synthetic RNA or a synthetic DNA) comprises only natural nucleobases, i.e., A, C, T and G in the case of a synthetic DNA, or A, C, T, and U in the case of a synthetic RNA.

As used herein, the term “encoding” refers to the inherent property of specific sequences of nucleotides in a polynucleotide, such as, for example, a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. The phrase “nucleotide sequence that encodes a protein or a RNA” may also include introns to the extent that the nucleotide sequence encoding the protein may in some version contain an intron(s).

As used herein, the “percent identity” between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the Needleman and Wunsch algorithm (Needleman, Saul B. & Wunsch, Christian D. (1970). “A general method applicable to the search for similarities in the amino acid sequence of two proteins”. Journal of Molecular Biology. 48 (3): 443-53). The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification. According to the invention a first amino acid sequence having at least 90% of identity with a second amino acid sequence means that the first sequence has 90; 91; 92; 93; 94; 95; 96; 97; 98; 99 or 100% of identity with the second amino acid sequence.

As used herein, the term “ANGPTL4” has its general meaning in the art and refers to the Angiopoietin-like protein 4 encoded by the ANGPTL4 gene. An exemplary human amino sequence for ANGPTL4 is SEQ ID NO:1. Human ANGPTL4 consists of 406 amino acids. Its protein structure is common to the angiopoietins, with a signal peptide directing secretion, an amino-terminal coiled-coil domain (CCD), a linker, and a carboxyterminal fibrinogen-like domain (FLD). ANGPTL4 undergoes processing, releasing CCD and FLD-containing fragments. The soluble CCD binds to lipoprotein lipase and converts the catalytically active dimeric form of the enzyme into inactive monomers, whereas the soluble FLD regulates vascular biology. The CCD fragments consists of the amino acid sequence which ranges from the amino acid residue at position 22 to the amino acid residue at position 170. The FLD fragment consists of the amino acid sequence which ranges from the amino acid residue at position 186 to the amino acid residue at position 406.

>sp|Q9BY76|ANGL4_HUMAN Angiopoietin-related protein 4
OS = Homo sapiens OX = 9606 GN = ANGPTL4 PE = 1 SV = 2
SEQ ID NO: 1
MSGAPTAGAALMLCAATAVLLSAQGGPVQSKSPRFASWDEMNVLAHGLLQLGQGLREHAE
RTRSQLSALERRLSACGSACQGTEGSTDLPLAPESRVDPEVLHSLQTQLKAQNSRIQQLF
HKVAQQQRHLEKQHLRIQHLQSQFGLLDHKHLDHEVAKPARRKRLPEMAQPVDPAHNVSR
LHRLPRDCQELFQVGERQSGLFEIQPQGSPPFLVNCKMTSDGGWTVIQRRHDGSVDFNRP
WEAYKAGFGDPHGEFWLGLEKVHSITGDRNSRLAVQLRDWDGNAELLQFSVHLGGEDTAY
SLQLTAPVAGQLGATTVPPSGLSVPFSTWDQDHDLRRDKNCAKSLSGGWWFGTCSHSNLN
GQYFRSIPQQRQKLKKGIFWKTWRGRYYPLQATTMLIQPMAAEAAS

As used herein, the term “subject”, “individual” or “patient” is used interchangeably and refers to any subject for whom diagnosis, treatment, or therapy is desired, particularly humans. Other subjects may include cattle, dogs, cats, guinea pigs, rabbits, rats, mice, horses, and the like. In some preferred embodiments, the subject is a human.

As used herein, the term “myocardial infarction” has its general meaning in the art and relates to the irreversible necrosis of the myocardium as a result of prolonged ischemia due to coronary thrombosis, i.e. the development of a clot in a major blood vessel serving the heart.

As used herein, the term “adverse post-ischemic cardiac remodeling” has its general meaning in the art and refers to the prominent changes that occur after myocardial infarction and that could be deleterious for the cardiac function. Cardiac remodeling involves molecular, cellular, and interstitial changes that manifest clinically as changes in size, shape, and function of the heart which occur after myocardial infarction. For instance, ventricular remodeling involves progressive enlargement of the ventricle with depression of ventricular function. Myocyte function in the myocardium remote from the initial myocardial infarction becomes depressed. In particular, adverse post-ischemic cardiac remodeling includes arrhythmias, cardiac dilation (assessed by left ventricular end diastolic volume indexed on body surface area or LVEDVi) and cardiac dysfunction (left ventricular ejection fraction or EF). Typically, adverse post-ischemic cardiac remodeling is defined as a >20% increase in left ventricular end-diastolic volume (LVEDV) at 6 months as compared to the initial evaluation.

As used herein, the term “cardiac fibrosis” has its general meaning in the art and refers to a condition characterized by the excessive production and deposition of extracellular matrix (ECM) proteins into the myocardium which leads to normal tissue architecture disruption, reduced tissue compliance, mechanical and electrical dysfunction. Following acute myocardial infarction, sudden loss of a large number of cardiomyocytes triggers an inflammatory reaction, ultimately leading to replacement of dead myocardium with a collagen-based scar.

As used herein, the term “heart failure” or “HF” has its general meaning in the art and embraces congestive heart failure and/or chronic heart failure. Functional classification of heart failure is generally done by the New York Heart Association Functional Classification (Criteria Committee, New York Heart Association. Diseases of the heart and blood vessels. Nomenclature and criteria for diagnosis, 6th ed. Boston: Little, Brown and co, 1964; 114). This classification stages the severity of heart failure into 4 classes (I-IV). The classes (I-IV) are: Class I: no limitation is experienced in any activities; there are no symptoms from ordinary activities; Class II: slight, mild limitation of activity; the patient is comfortable at rest or with mild exertion; Class III: marked limitation of any activity; the patient is comfortable only at rest; Class IV: any physical activity brings on discomfort and symptoms occur at rest.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “therapeutically effective amount” is meant a sufficient amount of the active ingredient for treating or reducing the symptoms at reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination with the active ingredients; and like factors well known in the medical arts. For example, it is well within the skill of the art to start doses of the compound at levels lower than those required to achieve the desired therapeutic effect and to gradually increase the dosage until the desired effect is achieved.

### Methods of Therapy:

The first object of the present invention relates to a method of treating adverse post-ischemic cardiac remodeling in a patient who experienced a myocardial infarction comprising administering to the patient a therapeutically effective amount of i) a polypeptide comprising an amino acid sequence having at least 90% of identity with the amino acid sequence ranging from the amino acid residue at position 186 to the amino acid residue at position 406 in SEQ ID NO: 1 or ii) a polynucleotide encoding thereof.

In particular, the method of the present invention is suitable for protecting against or reducing damage to the myocardium after a myocardial infarction, after, during or prior to ischemic reperfusion. More particularly, the method of the present invention is particularly suitable for reducing post ischemic left ventricular remodeling. Even more particularly, the method of the invention is suitable for increasing the left ventricle ejection fraction (LVEF), and/or for inhibiting left ventricle enlargement, and/or for reducing left ventricle end systolic volume, and/or reducing left ventricle end diastolic volume, and/or for ameliorating left ventricle dysfunction, and/or for improving myocardial contractibility.

In some embodiments, the method of the present invention is particularly suitable for preventing cardiac fibrosis.

The method of the present invention is also suitable for preventing heart failure in a patient who experienced a myocardial infarction.

In some embodiments, the polypeptide or polynucleotide of the present invention is administered to a patient having one or more signs or symptoms of acute myocardial infarction injury. In some embodiments, the patient has one or more signs or symptoms of myocardial infarction, such as chest pain described as a pressure sensation, fullness, or squeezing in the mid portion of the thorax; radiation of chest pain into the jaw or teeth, shoulder, arm, and/or back; dyspnea or shortness of breath; epigastric discomfort with or without nausea and vomiting; and diaphoresis or sweating.

In some embodiments, the polypeptide or polynucleotide of the present invention is administered simultaneously or sequentially (i.e. before or after) with a revascularization procedure performed on the patient. In some embodiments, the patient is administered with the polypeptide or polynucleotide of the present invention before, during, and after a revascularization procedure. In some embodiments, the patient is administered with the polypeptide or polynucleotide of the present invention as a bolus dose immediately prior to the revascularization procedure. In some embodiments, the patient is administered with the polypeptide or polynucleotide of the present invention continuously during and after the revascularization procedure. In some embodiments, the patient is administered with the polypeptide or polynucleotide of the present invention for a time period selected from the group consisting of at least 3 hours after a revascularization procedure; at least 5 hours after a revascularization procedure; at least 8 hours after a revascularization procedure; at least 12 hours after a revascularization procedure; at least 24 hours after a revascularization procedure. In some embodiments, the revascularization procedure is selected from the group consisting of percutaneous coronary intervention; balloon angioplasty; insertion of a bypass graft; insertion of a stent; directional coronary atherectomy; treatment with one or more thrombolytic agent(s); and removal of an occlusion.

In some embodiments, it is contemplated that the polypeptides of the invention are modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain. Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

In some embodiments, the polypeptide of the invention is fused a Fc domain of an immunoglobulin. Suitable immunoglobins are IgG, IgM, IgA, IgD, and IgE. IgG and IgA are preferred IgGs are most preferred, e.g. an IgG1. Said Fc domain may be a complete Fc domain or a function-conservative variant thereof. The polypeptide of the invention may be linked to the Fc domain by a linker. The linker may consist of about 1 to 100, preferably 1 to 10 amino acid residues.

According to the invention, the polypeptide of the invention may be produced by conventional automated peptide synthesis methods or by recombinant expression. General principles for designing and making proteins are well known to those of skill in the art. The polypeptides of the invention may be synthesized in solution or on a solid support in accordance with conventional techniques. Various automatic synthesizers are commercially available. The polypeptides of the invention may also be synthesized by solid-phase technology employing an exemplary peptide synthesizer such as a Model 433A from Applied Biosystems Inc. The purity of any given protein; generated through automated peptide synthesis or through recombinant methods may be determined using reverse phase HPLC analysis. Chemical authenticity of each peptide may be established by any method well known to those of skill in the art. As an alternative to automated peptide synthesis, recombinant DNA technology may be employed wherein a nucleotide sequence which encodes a protein of choice is inserted into an expression vector, transformed or transfected into an appropriate host cell and cultivated under conditions suitable for expression as described herein below. A variety of expression vector/host systems may be utilized to contain and express the peptide or protein coding sequence. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus, see Ghosh et al., 2002); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

In some embodiments, the polynucleotide of the present invention is a messenger RNA (mRNA).

In some embodiments, the polynucleotide is inserted in a vector, such a viral vector. As used herein, the term “viral vector” refers to a virion or virus particle that functions as a nucleic acid delivery vehicle and which comprises a vector genome packaged within the virion or virus particle. Typically, the vector is a viral vector which is an adeno-associated virus (AAV), a retroviral vector, bovine papilloma virus, an adenovirus vector, a vaccinia virus, or a polyoma virus. In some embodiments, the viral vector is a retroviral vector. As used herein, the term “retroviral vector” refers to a vector containing structural and functional genetic elements that are primarily derived from a retrovirus. In some embodiments, the retroviral vector of the present invention derives from a retrovirus selected from the group consisting of alpharetroviruses (e.g., avian leukosis virus), betaretroviruses (e.g., mouse mammary tumor virus), gammaretroviruses (e.g., murine leukemia virus), deltaretroviruses (e.g., bovine leukemia virus), epsilonretroviruses (e.g., Walley dermal sarcoma virus), lentiviruses (e.g., HIV-1, HIV-2) and spumaviruses (e.g., human spumavirus). In some embodiments, the retroviral vector of the present invention is a replication deficient retroviral virus particle, which can transfer a foreign imported RNA of a gene instead of the retroviral mRNA.

Thus the present invention encompasses use of virus-like particles. As used herein, the term “virus-like particle” or “VLP” refers to a structure resembling a virus particle but devoid of the viral genome, incapable of replication and devoid of pathogenicity. The particle typically comprises at least one type of structural protein from a virus. Preferably only one type of structural protein is present. Most preferably no other non-structural component of a virus is present. Thus, virus-like particles can be spontaneously self-assembled by viral structural proteins under appropriate conditions in vitro while excluding the genetic material and potential replication probability. virus-like particles, with a diameter of approximately 20 to 150 nm, also have the characteristics of nanometer materials, such as large surface area, surface-accessible amino acids with reactive moieties (e.g., lysine and glutamic acid residues), inerratic spatial structure, and good biocompatibility. Therefore, assembled virus-like particles have great potential as a delivery system for specifically carrying a variety of cargos. In some embodiments, one or more of the zinc finger motifs of the Gag protein is/are substituted by one or more RNA-binding domain(s). In some embodiments, the RNA-binding domain is the Coat protein of the MS2 bacteriophage, of the PP7 phage or of the Q3 phage, the prophage HK022 Nun protein, the ULA protein or the hPum protein. More preferably, the RNA binding domain is the Coat protein of the MS2 bacteriophage or of the PP7 phage. Even more preferably the RNA-binding domain is the Coat protein of the MS2 bacteriophage. These embodiments are particularly suitable for packaging the mRNA encoding for the ANGPTL4 FLD polypeptide into the VLP. Thus, in some embodiments, the mRNA encoding for the ANGPTL4 FLD polypeptide that is encapsuled in the virus particle of the present invention comprises at least one encapsidation sequence. By “encapsidation sequence” is meant an RNA motif (sequence and three-dimensional structure) recognized specifically by an RNA-binding domain as above described. Preferably, the encapsidation sequence is a stem-loop motif. Even more preferably, the encapsidation sequence of the retroviral particle is the stem-loop motif of the RNA of the MS2 bacteriophage or of the PP7 phage such as. The stem-loop motif and more particularly the stem-loop motif of the RNA of the MS2 bacteriophage or that of the RNA of the PP7 phage may be used alone or repeated several times, preferably from 2 to 25 times, more preferably from 2 to 18 times, for example from 6 to 18 times. In some embodiments, the present invention encompasses the use of the LentiFlash® technology that based on non-integrative lentiviral particles constructed using a bacteriophage coat protein and its cognate 19-nt stem loop, to replace the natural lentiviral Psi packaging sequence, in order to achieve active mRNA packaging into the lentiviral particles (Prel A, Caval V, Gayon R, Ravassard P, Duthoit C, Payen E, Maouche-Chretien L, Creneguy A, Nguyen T H, Martin N, Piver E, Sevrain R, Lamouroux L, Leboulch P, Deschaseaux F, Bouillé P, Sensébé L, Pagès J C. Highly efficient in vitro and in vivo delivery of functional RNAs using new versatile MS2-chimeric retrovirus-like particles. Mol Ther Methods Clin Dev. 2015 Oct. 21; 2:15039. doi: 10.1038/mtm.2015.39. PMID: 26528487; PMCID: PMC4613645).

In some embodiments, the vector of the present invention includes “control sequences”, which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell. Another nucleic acid sequence, is a “promoter” sequence, which is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

In some embodiments, the polypeptide or polynucleotide of the present invention can be conjugated to at least one other molecule. Typically, said molecule is selected from the group consisting of polynucleotides, polypeptides, lipids, lectins, carbohydrates, vitamins, cofactors, and drugs. In some embodiments, the polypeptide or polynucleotide of the present invention is formulated using one or more lipid-based structures that include but are not limited to liposomes, lipoplexes, or lipid nanoparticles (Paunovska, Kalina, David Loughrey, and James E. Dahlman. “Drug delivery systems for RNA therapeutics.” Nature Reviews Genetics (2022): 1-16). Liposomes are artificially-prepared vesicles which can primarily be composed of a lipid bilayer and can be used as a delivery vehicle for the administration of pharmaceutical formulations. Liposomes can be of different sizes such as, but not limited to, a multilamellar vesicle (MLV) which can be hundreds of nanometers in diameter and can contain a series of concentric bilayers separated by narrow aqueous compartments, a small unicellular vesicle (SUV) which can be smaller than 50 nm in diameter, and a large unilamellar vesicle (LUV) which can be between 50 and 500 nm in diameter. Liposome design can include, but is not limited to, opsonins or ligands in order to improve the attachment of liposomes to unhealthy tissue or to activate events such as, but not limited to, endocytosis. Liposomes can contain a low or a high pH in order to improve the delivery of the pharmaceutical formulations. As a non-limiting example, liposomes such as synthetic membrane vesicles are prepared by the methods, apparatus and devices described in US Patent Publication No. US20130177638, US20130177637, US20130177636, US20130177635, US20130177634, US20130177633, US20130183375, US20130183373 and US20130183372. In some embodiments, the liposomes are formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (as described in US20100324120) and liposomes which can deliver small molecule drugs such as, but not limited to, DOXIL® from Janssen Biotech, Inc. (Horsham, Pa.). The polypeptide of polynucleotide of the present invention can be encapsulated by the liposome and/or it can be contained in an aqueous core which can then be encapsulated by the liposome (see International Pub. Nos. WO2012031046, WO2012031043, WO2012030901 and WO2012006378 and US Patent Publication No. US20130189351, US20130195969 and US20130202684). In some embodiments, the polynucleotide of the present invention is formulated with stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104).

In some embodiments, the polypeptide or polynucleotide of the present invention is administered by intracoronary injection.

Typically, about less than 5, 10, 15, 20, 25, 30, 35, 40, 45, 50, 55, 60, 65, 70, 75, 80, 85, 90, 95 or 100 μg/kg of the polypeptide is administered as a bolus after the recanalization procedure by intracoronary injection. More particularly, about 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50, 51, 51, 53, 54, 55, 56, 57, 58, 59, or 60 μg/kg of the polypeptide is administered as a bolus after the recanalization procedure by intracoronary injection.

Typically the active ingredient of the present invention (i.e. the polypeptide or polynucleotide) is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The invention will be further illustrated by the following figures and examples. However, these examples and figures should not be interpreted in any way as limiting the scope of the present invention.

### FIGURES

FIG. 1. Comparison of infarct size expressed as the percentage of area at risk between control pigs and pigs injected with FLD at a concentration of 50 μg/kg as a bolus and subjected to 60 minutes of ischemia and 1 month of reperfusion. Panel a. shows infarct in n=6 pigs and panel b. shows quantification.

FIG. 2. Comparison of left ventricular function which assessed by electrocardiography at 3 days and 1 month after reperfusion in pigs injected with FLD vs. control animals.

FIG. 3. Comparison of left ventricle interstitial fibrosis assessed by Sirius red (a.) and Masson trichrome (b.) 1 month after reperfusion in pigs injected with FLD vs. control animals.

### EXAMPLE: ASSESSMENT OF THE CARDIOPROTECTIVE EFFECT OF FLD OF ANGPTL4 IN A PIG MODEL OF IR INJURY: TRANSLATIONAL ATTEMPT

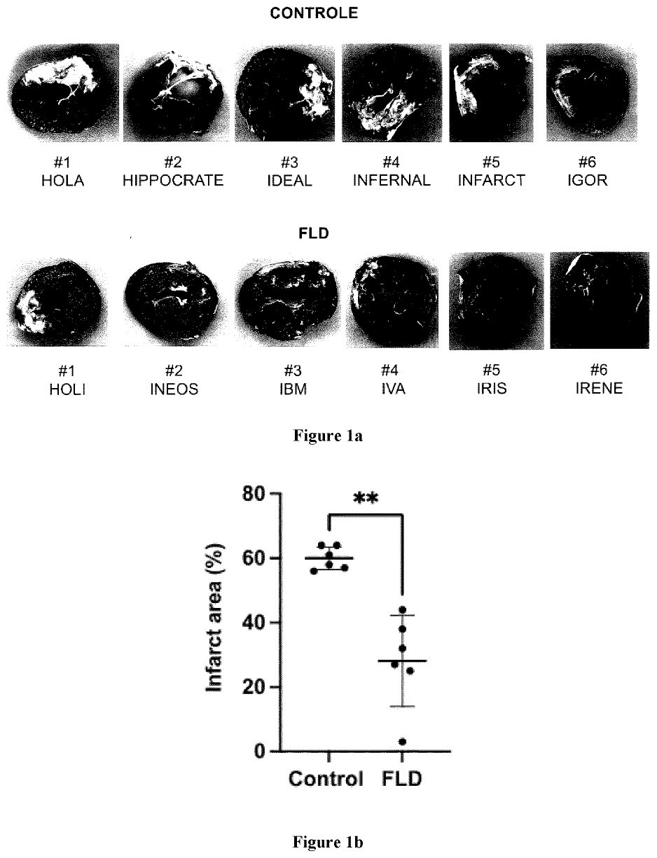

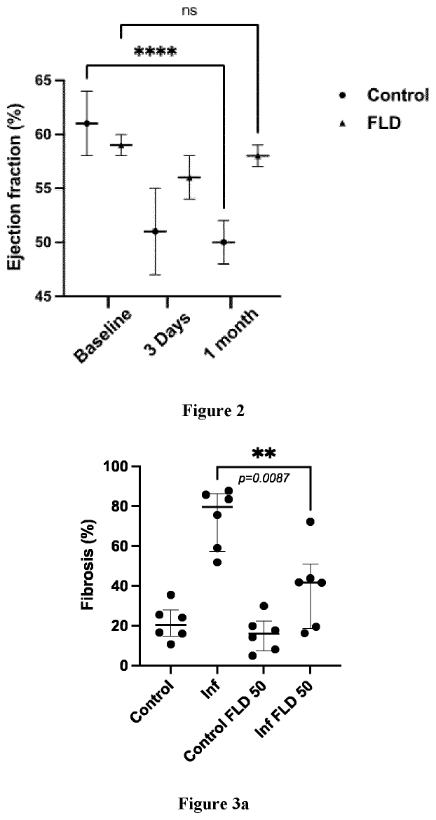

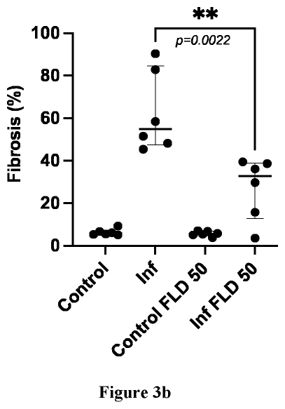

We previously showed that the therapeutic effects observed with ANGPTL4 in ischemic conditions are not due to the CCD fragments but are brought by the FLD fragment (WO2016/110498). The next step was to investigate FLD in a large animal model of myocardial infarction. As most of patients undergo today primary transcutaneous coronary intervention, we decided, as a translational attempt, to change the administration route and to inject FLD directly into the coronary circulation during the intracoronary permeabilization procedure. This will offer several advantages: i) to concentrate FLD to its target, i.e., the coronary circulation, ii) to reduce the amount of peripheral circulating FLD, i.e., to reduce the likelihood of non-cardiac effects and thus to reduce the occurrence of potential side and toxic effects, and iii) to simplify the protein administration scheme, i.e., only one intracoronary bolus at reperfusion without any further infusion and iv) to reduce the total amount of FLD needed per subject. FLD was thus administered after the recanalization procedure by intracoronary as a bolus of 50 μg/kg (Data not shown). As shown in FIG. 1a, infarct size measured using TTC/Evans Blue after 1 month and was conspicuously decreased by injection of 50 μg/kg FLD at reperfusion. We next quantified infarct size expressed as the percentage of area at risk and demonstrated a statistically significant reduction of infarct size between control pigs and pigs injected with FLD (FIG. 1b). This procedure also allowed us to measure left ventricular function which was assessed by electrocardiography at 3 days and 1 month after reperfusion demonstrating that ejection fraction was preserved in pigs injected with FLD vs. corresponding baseline significantly affected in control animals (FIG. 2). Interstitial fibrosis was also reduced in the FLD-treated hearts in the infarcted area (FIG. 3).

### REFERENCES

Throughout this application, various references describe the state of the art to which this invention pertains. The disclosures of these references are hereby incorporated by reference into the present disclosure.

1. A method of treating adverse post-ischemic myocardium remodeling in a patient who experienced a myocardial infarction comprising administering to the patient a therapeutically effective amount of i) a polypeptide comprising an amino acid sequence having at least 90% identity with the amino acid sequence ranging from the amino acid residue at position 186 to the amino acid residue at position 406 in SEQ ID NO:1 or ii) a polynucleotide encoding the polypeptide.

2. The method of claim 1 wherein the method reduces post ischemic left ventricular remodeling.

3. The method of claim 1 wherein the method prevents cardiac fibrosis in the patient who experienced a myocardial infarction.

4. The method of claim 1 wherein the method prevents heart failure in the patient who experienced a myocardial infarction.

5. The method according to claim 1, wherein the polypeptide or the polynucleotide is administered simultaneously or sequentially with a revascularization procedure performed on the patient.

6. The method of claim 5 wherein the polypeptide or the polynucleotide is administered to the patient as a bolus dose immediately prior to the revascularization procedure.

7. The method of claim 5 wherein the polypeptide or the polynucleotide is administered to the patient continuously during and after the revascularization procedure.

8. The method according to claim 1, wherein the polynucleotide is a messenger RNA (mRNA).

9. The method of according to claim 1, wherein the polynucleotide is present in a vector.

10. The method according to claim 1, wherein the polypeptide or the polynucleotide is be conjugated to at least one other molecule selected from the group consisting of polynucleotides, polypeptides, lipids, lectins, carbohydrates, vitamins, cofactors, and drugs.

11. The method according to claim 1, wherein the polypeptide or the polynucleotide is formulated using one or more lipid-based structures selected from the group consisting of liposomes, lipoplexes, or lipid nanoparticles.

12. The method according to claim 1, wherein about less than 5, 10, 15, 20, 25, 30, 35, 40, 45, 50, 55, 60, 65, 70, 75, 80, 85, 90, 95 or 100 μg/kg of the polypeptide is administered as a bolus by intracoronary injection after a recanalization procedure.

13. The method of claim 12 wherein 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50, 51, 51, 53, 54, 55, 56, 57, 58, 59, or 60 μg/kg of the polypeptide is administered.

14. The method of claim 2, wherein the method increases left ventricle ejection fraction (LVEF), and/or inhibits left ventricle enlargement, and/or reduces left ventricle end systolic volume, and/or reduces left ventricle end diastolic volume, and/or ameliorates left ventricle dysfunction, and/or improves myocardial contractibility.


SEQUENCE LISTING (1 sequence)

SEQ ID NO: 1 - protein, 406 aa, Homo sapiens
MSGAPTAGAALMLCAATAVLLSAQGGPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS